(19) United States
(12) Patent Application Publication
(10) Pub. No.: US 2017/0106039 A1
(43) Pub. Date: Apr. 20, 2017

(54) ANTI-ADDICTIVE COMPOSITION

(71) Applicants: Louise GUTOWSKI, Scottsdale, AZ (US); Walter GUTOWSKI, Scottsdale, AZ (US)

(72) Inventors: Dr. Louise Gutowski, Scottsdale, AZ (US); Walter Gutowski, Scottsdale, AZ (US)

(21) Appl. No.: 15/126,242

(22) PCT Filed: Mar. 16, 2015
(86) PCT No.: PCT/US15/20819

§ 371 Date: Sep. 14, 2016

Related U.S. Application Data
(60) Provisional application No. 61/953,596, filed on Mar. 14, 2014.

(51) Int. Cl. A61K 36/888; A61K 33/36; A61K 36/56; A61K 9/00; A61K 36/899; A61K 35/13; A61K 35/583; A61K 33/04; A61K 33/38; A61K 36/74

(52) U.S. Cl. CPC A61K 36/888; A61K 33/38; A61K 33/36; A61K 36/56; A61K 36/74; A61K 36/899; A61K 35/13; A61K 35/583; A61K 33/04; A61K 9/006

(57) ABSTRACT

Provided are combinations of homeopathic remedies to be used by persons inclined to addictive situations as well as methods for delivery of such preparations. The combination includes the nine remedies of Argentum Nitricum, Arsenicum Album, Avena Sativa, Caladium Seguinum, Carcinosin, Cinchona Officinalis, Lachesis Mutus, Nux Vomica and Sulphur. Alternatively, seven or eight of the remedies are included. Alternatively, Carcinosin is omitted. The quantity of each remedy is about one drop.

### TECHNICAL FIELD

This invention relates to homeopathic preparations of compounds as well as methods for delivery of such preparations and treatment of addictive conditions. In particular, the present invention provides for combinations of homeopathic remedies to be used by persons inclined to addictive situations.

### BACKGROUND

The World Health Organization (WHO) defines substance addiction as using a substance repeatedly, despite knowing and experiencing harmful effects. Substance addiction is a chronic, relapsing disease with loss of control over drug use, compulsive drug seeking and craving for a substance, use that persists despite negative effects, and physical and/or psychological dependence on the substance. Substance addiction typically follows a course of tolerance, withdrawal, compulsive drug taking behavior, drug seeking behavior, practice of addictive behavior, and relapse.

Substance abuse and addiction are public health issues with significant social and economic impact on both the addict and society by playing a major role in violent crime and the spread of infectious diseases. Examples of addictive substances include alcohol, caffeine, nicotine, cannabis (marijuana) and cannabis derivatives, opiates and other morphine-like opioid agonists such as heroin, phencyclidine and phencyclidine-like compounds, sedative hypnotics such as benzodiazepines and barbiturates and psychostimulants such as cocaine, amphetamines and amphetamine-related drugs such as dextroamphetamine and methylamphetamine

Alcohol is one of the most commonly abused substances at a global level. Additionally, alcoholism leads to serious liver and cardiovascular disease and generates dependence resulting in severe mental disorders, social problems and adverse consequences including the division of families, tragic accidents and the reduction of work performance. According to the WHO, alcohol consumption is responsible for 20-30% of esophageal and liver cancer, liver cirrhosis, homicides, epilepsy, and motor vehicle accidents worldwide.

Globally, alcohol abuse leads to about 1.8 million deaths per year. Compulsive behavior towards the consumption of alcohol is a core symptom of the disorder. Numerous approaches have been investigated to help alcoholic patients to control alcohol drinking and also alcohol cravings and relapse.

Numerous medications such as naltrexone, acamprosate, ondansetron, disulfiram, gamma hydroxybutyrate (GHB), and topiramate have been tested for their potential therapeutic effect on alcohol abuse. Few of these, such as naltrexone, acamprosate, and disulfiram, have received FDA approval for alcoholism. Among these medications, the non-selective opioid antagonist naltrexone is currently the pharmacological best option. However, despite some promising results none of these medications, including naltrexone, is of sufficient efficacy in alcoholism and prognosis remains poor.

Nicotine is one of the most widely used addictive drugs, and nicotine abuse is the most common form of substance abuse. The WHO estimates that there are 1.25 billion smokers worldwide, representing one third of the global population over the age of 15. The WHO further estimates that 5 million deaths occur annually as a direct result of tobacco use, making nicotine abuse the largest single preventable cause of death worldwide. In industrialized countries, 70-90% of lung cancer, 56-80% of chronic respiratory disease, and 22% of cardiovascular disease instances are attributed to nicotine addiction. Cigarette smoking is associated with 430,000 deaths per year in the US alone and is estimated to cost the nation 80 billion dollars yearly in health care costs. Tobacco use accounts for one third of all cancers, including cancer of the lung, mouth, pharynx, larynx, esophagus, cervix, kidney, ureter, and bladder. An estimated 20% of the deaths from heart disease are attributable to smoking. Expectant women who smoke are at greater risk than nonsmokers for premature delivery, spontaneous abortion, and infants with decreased birth weight.

Nicotine use results in increased levels of the neurotransmitter dopamine, which activates the reward pathways to regulate feelings of pleasure and to mediate the desire to consume nicotine. Symptoms associated with nicotine withdrawal include craving, irritability, anger, hostility, aggression, fatigue, depression, and cognitive impairment, which lead the abuser to seek more nicotine. Environmental conditioning factors and exposure to psychological stress represent additional factors motivating nicotine use in smokers. Repeated nicotine use results in the development of tolerance, requiring higher doses of nicotine to produce the same initial stimulation.

Most therapies developed for nicotine addiction have modest success in preventing relapse, leading to a high failure rate in attempts to quit smoking. Treatments include the use of nicotine replacement products, anti-depressants, anti-hypersensitives, and behavioral therapy.

Psychostimulants, such as cocaine and amphetamines, temporarily cause euphoria, increased alertness, and increased physical capacity in humans. These substances first increase dopamine transmission, but long term drug usage results in a reduction of dopamine activity, leading to dysregulation of the brain reward system and dysphoria. The WHO estimates 33 million people around the world abuse amphetamines.

Chronic cocaine abuse can result in hyperstimulation, tachycardia, hypertension, mydriasis, muscle twitching, sleeplessness, extreme nervousness, hallucinations, paranoia, aggressive behavior, and depression. Cocaine overdose causes tremors, convulsions, delirium, and death resulting from heart arrhythmias and cardiovascular failure. Desipramine, amantadine and bromocriptine have been shown to decrease cocaine withdrawal symptoms.

Several lines of evidence suggest that molecular mechanisms underlying relapse to addiction are common to different classes of drugs of abuse. Drug craving and loss of control over drug taking behavior associated with relapse are under the direct influence of stress and environmental conditioning stimuli, the two major factors affecting resumption to drug use.

Chronic drug abuse produces neuroadaptive changes not only within systems implicated in the acute reinforcing effects of ethanol, but also within other motivational systems, notably brain stress-regulatory mechanisms. Stress initiates and maintains drug abuse, and is a major cause of relapse. Stressful stimuli also elicit return to cocaine, heroin, and ethanol-seeking behavior in drug-free animals following extinction and these findings provide experimental support for a role of stress in relapse.

Stress-related drug-seeking behavior was alleged to be mediated via activation of the hypothalamic-pituitary-adrenal (HPA) axis. However, growing evidence suggests that the non-neuroendocrine corticotropin-releasing factor (CRF) system in the central nucleus of the amygdala (CeA) separately regulates addictive behavior associated with stress. The CeA is rich in CRF immunoreactive cell bodies, terminals, and receptors, and this neuronal CRF system has been implicated in the mediation of behavioral and emotional responses to stressful stimuli. For example, immobilization stress elevates extracellular CRF levels in the CeA while intra-CeA injection of a CRF receptor antagonist reduces anxiety produced by social and environmental stressors. Anxiety and stress-like symptoms are central to drug and alcohol withdrawal syndromes. Considering the evidence of a role of CRF neurons in the CeA in the regulation of emotional and anxiogenic effects of stress, it is likely that anxiogenic and stress-like consequences of withdrawal from drugs of abuse are mediated by the CRF system in the CeA as well.

Changes in the regulation of the activity of the CRF system within the CeA may represent a critical neuroadaptive mechanism responsible for the development of dependence and compulsive drug-seeking behavior.

In humans, relapse risk involves several causes. For example, drug cues increase vulnerability to relapse, particularly with protracted withdrawal symptoms. Stress also exacerbates relapse risk. Recent work addressing these issues has confirmed that additive interactions between the response-reinstating effects of ethanol-associated cues and stress can indeed be demonstrated, and that these effects are enhanced in rats with a history of ethanol dependence.

A major unsolved problem today is addiction treatment. Drugs are not optimal and the few successful methods involve removing the affected individual from society for months to engage in talk therapy and learn new habits. Few can afford this.

### SUMMARY

In a preferred embodiment, there is disclosed a homeopathic composition for treating addictions, the composition consisting of Argentum Nitricum, Arsenicum Album, Avena Sativa, Caladium Seguinum, Carcinosin, Cinchona Officinalis, Lachesis Mutus, Nux Vomica and Sulphur. Optionally the quantities of each remedy are about one drop. Optionally, the individual remedies have been diluted to 30 C.

In another embodiment a homeopathic composition for treating addictions consists of at least seven of the following remedies: Argentum Nitricum, Arsenicum Album, Avena Sativa, Caladium Seguinum, Carcinosin, Cinchona Officinalis, Lachesis Mutus, Nux Vomica and Sulphur. Optionally the quantities of each remedy are about one drop. Optionally, the individual remedies are diluted to 30 C.

In another embodiment, a homeopathic composition for treating addictions comprises the remedies Argentum Nitricum, Arsenicum Album, Avena Sativa, Caladium Seguinum, Cinchona Officinalis, Lachesis Mutus, Nux Vomica and Sulphur. Optionally the quantities of each remedy are about one drop. Optionally the individual remedies are diluted to 30 C.

### BRIEF DESCRIPTION OF THE FIGURES

FIGS. 1A and 1B are tables showing exemplary compositions of the invention.

FIG. 2 shows the initial and two-month patient reports of addictive substance use.

FIG. 3 shows the initial and two-month patient reports of addictive substance cravings that can be precursors to usage.

### DETAILED DESCRIPTION

I have consulted with many recovering addict patients. Over time, I have prescribed various individual remedies which worked well initially. However, when I tried known remedies, they did not benefit my patients for very long. I started a series of efforts to develop a more effective and long-lasting homeopathic treatment. I combined various products in an effort to help patients to maintain their sobriety for longer periods.

We eventually invented a composition of nine homeopathic remedies for my anti-craving composition. They are Argentum Nitricum 30 C, Arsenicum Album 30 C, Avena Sativa 30 C, Caladium Seguinum 30 C, Carcinosin 30 C, Cinchona Officinalis 30 C, Lachesis Mutus 30 C, Nux Vomica 30 C and Sulphur 30 C. Each of these will be discussed independently.

Argentum Nitricum 30 C is also known as silver nitrate, nitric acid silver and traditionally as lunar caustic. It is an inorganic compound that decomposes to elemental silver. Silver salts are known to have antiseptic properties. Dentists sometimes use silver nitrate in swabs to heal oral ulcers. Podiatrists kill cells in the nail bed with this compound. It is also used to cauterize nose bleeds. It has some success against warts. However, I could find no record of use in addictions.

Arsenicum Album 30 C is a homeopathic treatment for gastrointestinal problems due to eating too much fresh fruit and vegetables, drinking too much alcohol and food poisoning. Associated symptoms get better, too, including loss of appetite, indigestion, diarrhea, vomiting and dehydration. Arsenicum Album is also used for cold weather conditions including asthma, inflamed eyes and headaches. It also may be employed in treating fear and anxiety accompanied with chills, restlessness, and fatigue. It is extracted from arsenic oxide from the mineral ore arsenopyrite. It is usually taken orally as a tablet, pellets, powder, granules or liquid.

Avena Sativa 30 C is considered a nerve and brain tonic. It is a herb obtained from oats (genus Avena Sativa) and includes compounds which are both sedative and soothing. It is used in cases of high cholesterol, alcoholism, debility, drug addictions and abuse, impotency, insomnia, neurasthenia, anxiety and nervous exhaustion. It is said to be useful when an individual cannot focus on any one subject. It is available in capsule form, in a tincture or as a tea. Dosage varies depending on the reasons for taking the remedy.

Caladium Seguinum 30 C is also known as American Arum or Cal for short. It is said to have a marked action on the genital organs and pruritus in that region and modifies tobacco craving and asthma complaints and a host of other complaints. Some homeopaths believe Caladium Seguinum reduces cravings.

Carcinosin 30 C is a homeopathic composition prepared from cancerous tissues typically taken from the breast. It is used in patients with cancer and in non-cancerous patients having a strong family history of cancer. It is for the introverted who have a propensity to be obsessive and workaholics, resulting in fatigue and other ailments. It has been recommended for individuals with a strong desire for foods rich in fat content, particularly butter and chocolate. It has also been used in chronic fatigue syndrome, insomnia, respiratory ailments, abdominal pain, and skin blemishes and growths. There is allegedly a Carcinosin personality with addiction to drugs, alcohol, caffeine and cigarettes, as well as sensitivity towards foods.

In the US the FDA has asked that Carcinosin not be used in products for human consumption. Therefore, we contemplate compositions lacking Carcinosin.

Cinchona Officinalis 30 C is also called Peruvian bark, Jesuit's bark, red bark and quinine bark. Cinchona Officinalis is native to the Amazon rainforest and one of its ingredients, quinine, has long been used by mainstream medicine to treat malaria. It helps increase appetite, treats bloating, fullness and other stomach problems, hemorrhoids, varicose veins and leg cramps, enlarged spleen and cancer. Some homeopaths believe it helps to ease withdrawal symptoms.

Lachesis Mutus 30 C is derived from Bushmaster snake venom and makes blood more fluid. It has been used for head and mind ailments such as hot flashes, delirium tremens with trembling and confusion, tension, restlessness, pain through head, facial neuralgia, craving for alcohol and oysters.

Nux Vomica 30 C comes from Strychnos nux-vomica but colloquial names include poison nut, quaker button, dog button, vomit nut and vomit weed. The seed of the strychnine tree, native to India and other parts of Asia, is believed by homeopaths to combat withdrawal symptoms.

Sulphur 30 C, also called flowers of sulfur and brimstone, is a homeopathic remedy used to treat skin rash worsened by heat and water. It also has been used for digestion problems, women's symptoms of depression, memory problems and menopausal irritability. It also is used for respiratory problems like coughs associated with colds.

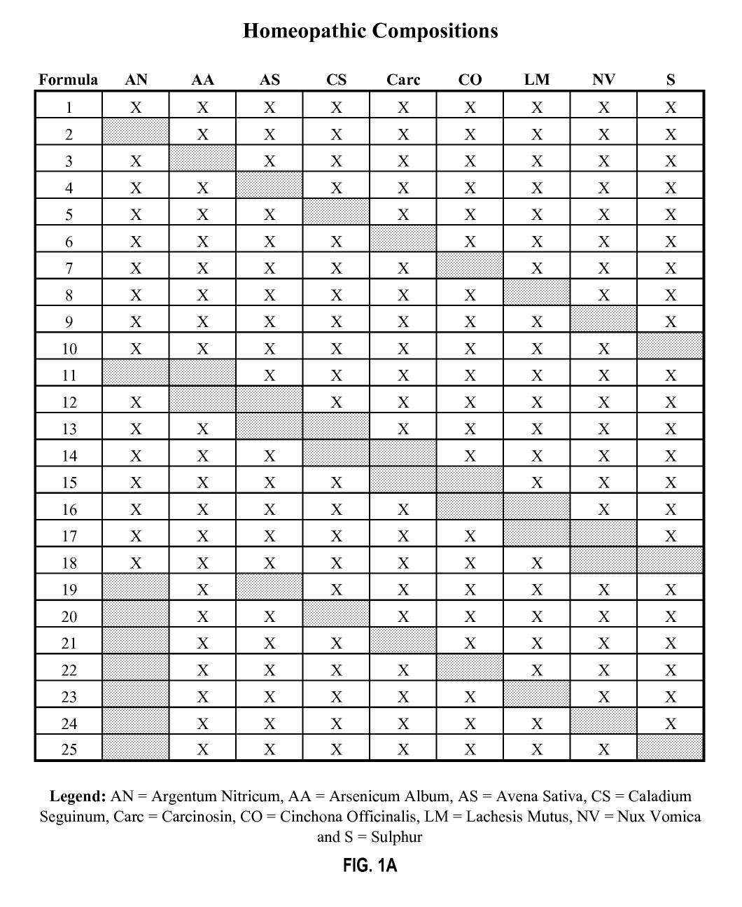

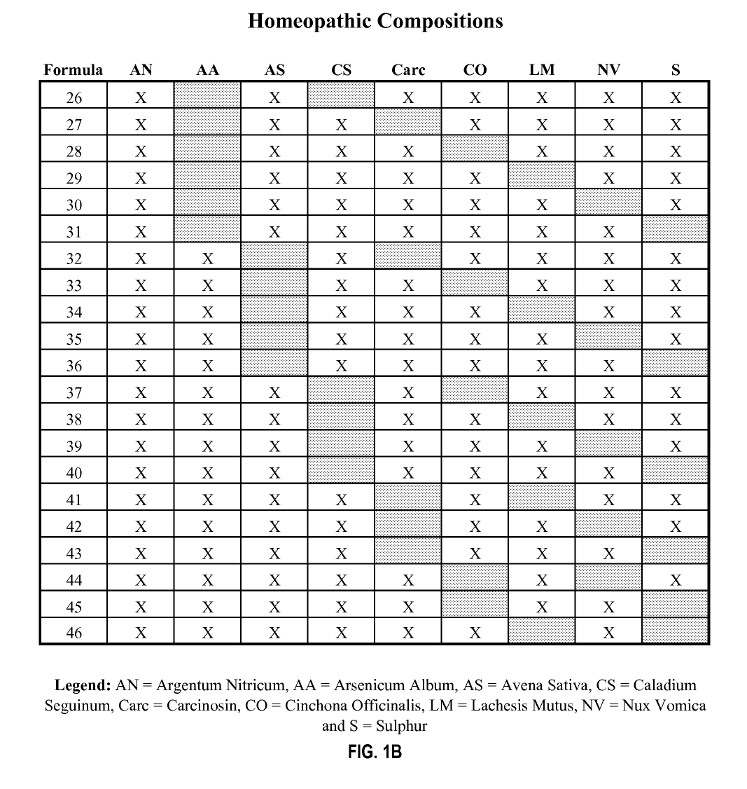

I added one drop of each remedy in the formula to a 1 oz bottle and filled the bottle with water, preferably filtered water. See FIGS. 1A and 1B for exemplary compositions. To mix the composition, I use succession which is pounding the bottle into the palm of my hand repeatedly 25-40 times.

Water can be any of purified, deionized and/or mineral water. Preservatives include but are not limited to ethanol, citric acid and potassium sorbate. Ethanol is generally used in 10% to 25% amounts.

For each dosage, I instructed the patient to place 8-10 drops under the tongue and hold it there for 20-30 seconds. The recommended dosage is two times per day in the mid-morning and mid-afternoon. Alternatively, the composition may be taken between meals at least 20 minutes before or after a meal.

Thus, the present invention includes methods of treating or preventing an addiction, comprising administering one or more PDE7 inhibitors to a subject having an addiction or at risk for developing an addiction. In various embodiments, the subject is addicted to an addictive agent or behavior, including, but not limited to, any of the addictive agents and behaviors described herein. The subject may be physically or physiologically dependent on the substance or behavior; the subject may be psychologically dependent; or the subject may be both physically and psychologically dependent. The subject may be addicted to one or more than one addictive agent or behavior.

The above described remedies can be used in any combination, as illustrated in FIGS. 1A and 1B. All nine remedies are preferred. However, seven or eight compositions are contemplated. FIGS. 1A and 1B are for illustrative purposes only and is not intended to be limiting.

Various combinations are also contemplated as included in the instant invention, such as those including not just all nine constituents, but also various combination of 5, 6, 7 or 8 remedies.

### EXAMPLE 1

### Multiply Addicted Person

A 38-year-old stocky, Caucasian male presented with a low back injury following an auto accident. He was given the indicated chiropractic and naturopathic therapy for muscle and ligament injuries. His history indicated multiple addictions and continuing cravings for the addictive substances after participation in rehabilitation programs.

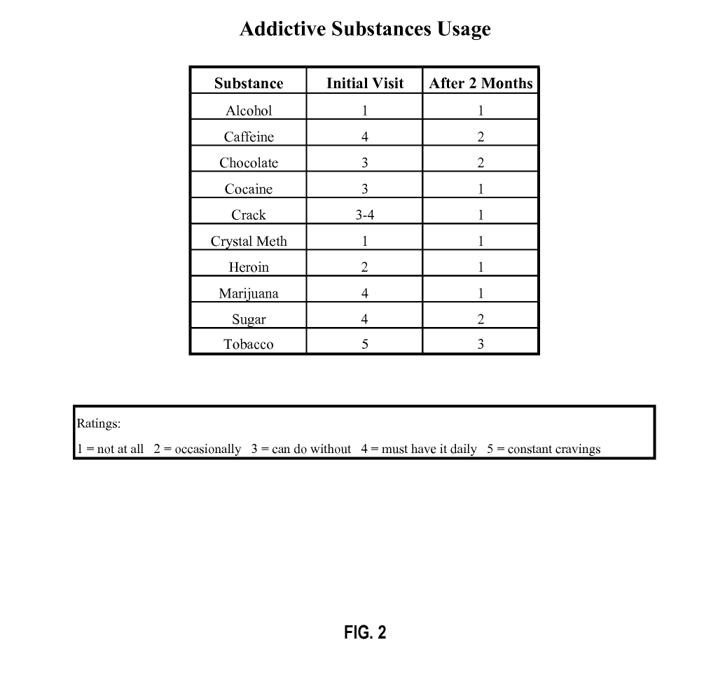

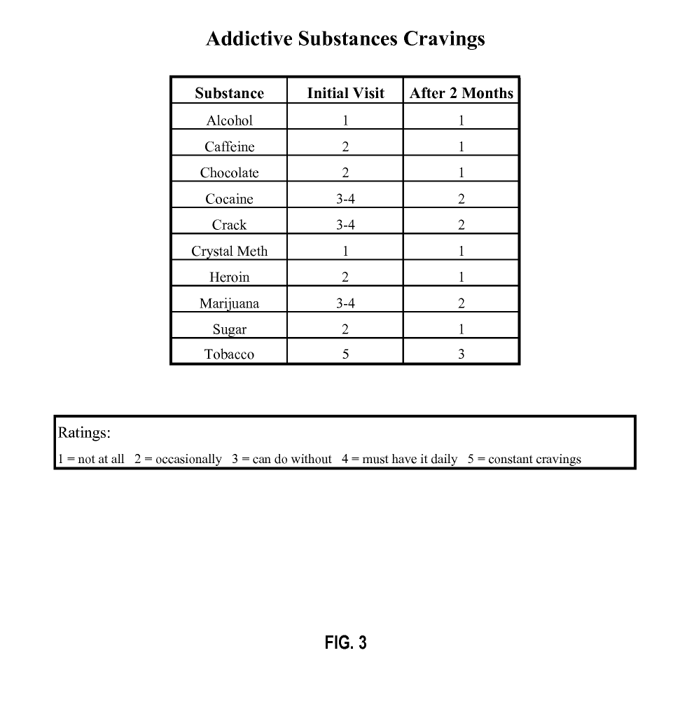

As shown in FIG. 2, he initially reported usage of a wide variety of addictive substances, including caffeine, chocolate, cocaine, crack, heroin, marijuana, sugar and tobacco but not alcohol or crystal meth. As shown in FIG. 3, he initially had cravings primarily for cocaine, crack and marijuana and lesser cravings for heroin, caffeine, chocolate and sugar.

He was given his first bottle of the inventive nine-remedy anti-addiction/craving homeopathic composition. He stated he took the composition intermittently for about one week and then lost the bottle. He received a replacement bottle and reported taking the product according to the directions (10 drops mid-morning and mid-evening).

At two months after receiving the replacement bottle, he completed the questionnaire regarding drug usage and cravings and the results are shown in FIGS. 2 and 3. He reported that usage of all substances had decreased. At two months he reported no use of alcohol, cocaine, crack, crystal meth, heroin and marijuana. His use of caffeine, chocolate and sugar decreased, as shown in FIG. 2.

At two months he reported that cravings remained absent for alcohol and crystal meth and were decreased for caffeine, chocolate, cocaine, crack, heroin, marijuana, sugar and tobacco.

He was sober for four months after receiving the replacement bottle. He then relapsed and entered rehabilitation. Upon discharge in two weeks, he started using again for about five days but stopped. On a subsequent office visit, he received another bottle of the inventive nine-remedy composition. He reported being clean and sober since receiving that last bottle two years and three months ago.

In a recent telephone interview, the patient reported pride that he had been sober for over two years. He said that the rehabilitation programs did not seem to help, but the naturopathic regimen had helped him to be “clean and sober.” Specifically he had no more drug use or cravings.

To summarize, this patient illustrates a remarkable decrease in both addictive use and cravings for weeks then years.

As used herein, unless the context makes clear otherwise, “treat,” and similar words such as “treatment,” “treating” etc., is an approach for obtaining beneficial or desired results, including and preferably clinical results. Treatment can involve optionally either the reducing or amelioration of a disease or condition, (e.g., addiction or relapse use or behavior), or the delaying of the progression of the disease or condition (e.g., addiction, or relapse use or behavior).

As used herein, unless the context makes clear otherwise, “prevent,” and similar words such as “prevention,” “preventing” etc., is an approach for preventing the onset or recurrence of a disease or condition, (e.g., addiction, or relapse use or behavior) or preventing the occurrence or recurrence of the symptoms of a disease or condition, or optionally an approach for delaying the onset or recurrence of a disease or condition or delaying the occurrence or recurrence of the symptoms of a disease or condition.

According to certain embodiments of the present invention, a subject is provided with our inventive mixture in combination with an addictive therapeutic agent, with the dosage of the addictive therapeutic agent being determined to achieve the desired therapeutic effect and the dosage of my mixture being determined to eliminate or reduce the potential for addiction to the addictive therapeutic agent.

In particular embodiments, a subject is considered at risk of addiction or relapse to use of an addictive agent or practice of an addictive behavior when the subject has previously been addicted to the same or a different addictive agent or addictive or compulsive behavior. In certain embodiment, the subject is considered at risk of addiction or relapse to use of an addictive agent or practice of an addictive behavior when the subject is psychologically addicted to an addictive agent or addictive or compulsive behavior, even if the subject is no longer physically addicted. In one embodiment, the addictive behavior is binge eating. Subjects at risk of binge eating typically have at least one of the following in their history: recurring food restrictions or yo-yo dieting, eating in response to environmental stress, preference for highly palatable and high caloric food, eating after reaching fullness, and eating to the point of discomfort. In another embodiment, the subject suffers from a primary impulse-control disorder.

The term “addiction” is used to describe a recurring compulsion by an individual to engage in some specific activity, despite harmful consequences to the individual's health, mental state or social life. The term is often reserved for drug addictions, but it is applied to other compulsions, such as problem gambling, binge eating, and sugar “cravings”. Factors that have been suggested as causes of addiction include genetic, biological/pharmacological and social factors.

The medical community now makes a careful theoretical distinction between physical or physiological dependence (characterized by symptoms of withdrawal) and psychological dependence (sometimes referred to simply as addiction). Addiction is now narrowly defined as “uncontrolled, compulsive use.” If there is no harm being suffered by, or damage done to, the patient or another party, then clinically it may be considered compulsive, but to the definition of some it is not categorized as “addiction.” In practice, the two kinds of addiction (physiological dependence and psychological dependence) are not always easy to distinguish. Addictions often have both physical and psychological components.

“Physical dependence” (or “drug dependence”) refers to a state resulting from habitual use of a drug, where negative physical withdrawal symptoms result from abrupt discontinuation. Examples of addictive agents for which a user may develop a physical dependence include nicotine, opioids, barbiturates, benzodiazepines, and alcohol, i.e., ethyl alcohol, GHB, and methaqualone.

Commonly abused stimulants such as cocaine or amphetamine class drugs are not believed to cause significant physical dependence. However, their potential for extreme psychological addiction can compel the user to consume amounts which become physically damaging, but life-threatening withdrawal effects have not been observed.

Sugar is a highly refined substance that does not appear alone in nature. It looks a lot like cocaine, and sugar acts a lot like heroin when it hits the brain. Although the idea that sugar was addictive was controversial among scientists for years, they began to take note when the paper titled Sugar and Fat Bingeing Have Notable Differences in Addictive-Like Behavior was published in the Journal of Nutrition in 2009.

The study showed that sugar affects the brain chemistry and thus might be expected to cause addictive behavior. In the study, written by Nicole Avena and others, it was shown that sugar bingeing can cause withdrawal symptoms and cravings.

The behavioral effects are similar to the neurochemical changes in the brain that also occur with addictive drugs. One finding of that study is seldom discussed—both sugar and other sweet tasting substances activate beta endorphin receptor sites in the brain, the same receptor sites that are activated by heroin and morphine.

As used herein, “addictive agent(s)” includes any and all agents to which a subject can become addicted, either physically or psychologically, or both. As noted above, addiction includes addiction to chemical entities, such as drugs, e.g., ethyl alcohol, nicotine, or cocaine, as well as addiction to other behaviors, e.g., binge eating disorder, pathological gambling, pathological use of electronic devices, e.g., BlackBerry®, pathological use of electronic video games, pathological use of electronic communication devices, pathological use of cellular telephones, addiction to pornography, sex addiction, obsessive-compulsive disorder, compulsive spending, intermittent explosive disorder, kleptomania, pyromania, trichotillomania, compulsive over-exercising, and compulsive overworking.

As used herein “binge eating disorder” or “binge eating” includes at least one of the following symptoms: eating large amounts of food, eating even when full, rapid eating, feeling that eating behavior is out of control, eating substantial amounts of food when not hungry, frequent dieting possibly without weight loss, eating alone, feeling depressed or disgusted about eating habits and eating in response to stress. Binge eating disorder is distinct from bulimia and binge purge syndromes.

In one embodiment, the addictive agent is alcohol and the additional therapeutic agent is an opioid antagonist or a mixed opioid antagonist/partial agonist. In a particular embodiment, the opioid antagonist is naltrexone. In another embodiment, the mixed opioid partial agonist/antagonist is buprenorphine.

In one embodiment, the addictive agent is alcohol, and the additional therapeutic agent is topiramate or levetiracetam.

For the purposes of promoting an understanding of the principles of the invention, reference will now be made to the exemplary embodiments illustrated in the drawings, and specific language will be used to describe the same. It will nevertheless be understood that no limitation of the scope of the invention is hereby intended. Any alterations and further modifications of the inventive features illustrated herein, and any additional application of the principles of the invention as illustrated herein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus appearances of the phrases an “embodiment,” and “example,” and similar language throughout this specification may, but do not necessarily, all refer to the same embodiment, to different embodiments, or to one or more of the figures. Additionally, reference to the words “embodiment,” “example” or the like for two or more features, elements, etc., does not mean that the features are necessarily related, dissimilar, the same, etc.

Each statement of an embodiment or example is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional un-recited elements or method steps. “Comprising” is to be interpreted broadly and including the more restrictive terms “consisting of” and “consisting essentially of.”

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage or characteristic described in connection with an embodiment is included in at least one embodiment of the present invention. Thus, discussion of the features and advantages, and similar language, throughout this specification may, but does not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description or may be learned by the practice of the invention as set forth.

1. A homeopathic composition for treating addictions, the composition consisting of the remedies Argentum Nitricum, Arsenicum Album, Avena Sativa, Caladium Seguinum, Carcinosin, Cinchona Officinalis, Lachesis Mutus, Nux Vomica and Sulphur.

2. The homeopathic composition of claim 1 wherein the quantities of each remedy are about one drop.

3. The homeopathic composition of claim 1 wherein the individual remedies are at dilutions of 30 C.

4. A homeopathic composition for treating addictions, the composition consisting of at least seven of the following remedies: Argentum Nitricum, Arsenicum Album, Avena Sativa, Caladium Seguinum, Carcinosin, Cinchona Officinalis, Lachesis Mutus, Nux Vomica and Sulphur.

5. The homeopathic composition of claim 4 wherein the remedy quantities are about one drop.

6. The homeopathic composition of claim 4 wherein the individual remedies are at dilutions of 30 C.

7. A homeopathic composition for treating addictions, the composition comprising the remedies of Argentum Nitricum, Arsenicum Album, Avena Sativa, Caladium Seguinum, Cinchona Officinalis, Lachesis Mutus, Nux Vomica and Sulphur.

8. The homeopathic composition of claim 7 wherein the quantities are about one drop of each.

9. The homeopathic composition of claim 7 wherein the individual remedies are at dilutions of 30 C.
